(19) United States
(12) Patent Application Publication
(10) Pub. No.: US 2024/0279098 A1
(43) Pub. Date: Aug. 22, 2024

(54) SYSTEMS AND METHODS FOR BIOREMEDIATION OF PER- AND POLYFLUOROALKYL SUBSTANCES AND 1,4-DIOXANE

(71) Applicant: The Trustees of Columbia University in the City of New York, New York, NY (US)

(72) Inventor: Raymond SAMBROTTO, Blauvelt, NY (US)

(73) Assignee: The Trustees of Columbia University in the City of New York, New York, NY (US)

(21) Appl. No.: 18/640,659

(22) Filed: Apr. 19, 2024

Related U.S. Application Data
(63) Continuation of application No. PCT/US22/78425, filed on Oct. 20, 2022.
(60) Provisional application No. 63/270,543, filed on Oct. 21, 2021.

(51) Int. Cl. C02F 3/34; B09C 1/10; C12N 1/20; C12R 1/07

(52) U.S. Cl. CPC C02F 3/34; B09C 1/10; C12N 1/205; C12R 2001/07

(57) ABSTRACT

Systems and methods for performing bioremediation using the Geobacillus sp. bacterial strain are provided which are capable of effectively degrading 1,4-dioxane and/or per- and polyfluoroalkyl substances (PFAS) and/or BaP and efficiently degrading other ring-based organic contaminants. The exemplary bioremediation methods include steps for administering an effective, degrading amount of the bacteria to soil containing excess amounts of 1,4-dioxane and/or PFAS and/or BaP and incubating the bacteria administered to the soil at a given temperature and for a duration that are suitable for promoting incubation and reducing and the concentration of 1,4-dioxane and/or PFAS and/or BaP below a maximum concentration, as might be specified by a regulatory body.

### CROSS REFERENCE TO RELATED APPLICATIONS

The present application is a Continuation of PCT/US22/78425, filed Oct. 20, 2022, which claims priority to U.S. patent application No. 63/270,543, filed Oct. 21, 2021, both of which are hereby incorporated by reference in their entireties.

### GOVERNMENT LICENSE RIGHTS

This invention was made with government support under grant 1927687 awarded by the National Science Foundation. The government has certain rights in the invention.

### FIELD OF THE INVENTION

The present invention relates to the field of environmental restoration techniques for soils and sediments contaminated with toxic organic pollutants and, more specifically systems and methods for enhanced remediation of contaminated materials using a Geobacillus sp. bacterial strain or an enhanced variant thereof.

The present invention also relates to the methods for the bioremediation of sorbent substances, such as soils, sediments and sorbent materials (e.g., commercial sorbent materials), and water and groundwater, contaminated with 1,4-dioxane and/or per- and polyfluoroalkyl substances (PFAS) and, more specifically systems and methods for enhanced remediation of contaminated materials using a Geobacillus sp. bacterial strain or an enhanced variant thereof.

### BACKGROUND OF THE INVENTION

Human industrial activities generate a large amount and diversity of pollutant and toxic compounds. Among these are the carcinogenic polycyclic aromatic hydrocarbons (PAHs) and per- and polyfluoroalkyl substances (PFAS), a family of synthetic industrial chemicals that includes perfluorooctanoic acid (PFOA), perfluorooctane sulfonate (PFOS), GenX (the ammonium salt of hexafluoropropylene oxide dimer acid (HFPO-DA) fluoride), and many other chemicals. PFAS have been produced since the 1940s and are now found in soil and groundwater sites throughout the U.S. and have been classified by the Environmental Protection Agency (EPA) as a health hazard to humans by all routes of exposure. PFOA and PFOS are persistent in the environment and accumulate in the human body. Their use has been so ubiquitous that the vast majority of the global human population has measurable levels of PFAS in their tissues. PFAS have been shown to cause adverse health effects such as low infant birth weights and effects on the immune system. PFOA has been implicated in human cancer and PFOS has been shown to disrupt human thyroid function.

Sources of PFAS include food packaging, commercial household products such as stain resistant fabrics and non-stick products such as Teflon and fire-fighting foams. PFAS have been shown to contaminate drinking water, especially in areas with a production facility that uses PFAS, landfills and firefighting training facilities, military aircraft facilities and civilian airports. The most acute problems with PFAS contamination in the environment are associated with airports and fire training facilities that have used large amounts of these chemicals as part of their routine training programs. PFAS are water soluble and migrate through the soil to contaminate groundwater. A prominent example is the groundwater contamination associated with historical releases from Wurtsmith Air Force Base in Iosco County Michigan. Here the PFAS plume has reached nearby Lake Huron.

The remediation of soils and sediments contaminated with PAHs typically depend on landfills or incineration. Few options exist to remove PFAS from groundwater. Current solutions include erecting barriers with Portland cement and absorbants such as Cetco's Fluoro-Sorb that bind to PFAS to immobilize the PFAS in the soil layer, containing the PFAS and preventing migration into the groundwater, but not degrading it. Other solutions include pumping and treating the contaminated groundwater with ion exchange resin. This resin is a secondary waste stream that is contaminated with PFAS and is typically stored above ground. This treatment removes the PFAS from groundwater but does not degrade it.

In some cases, the resin can be regenerated by solvent washing to yield an eluate of solvent containing high concentrations of PFAS. This contaminated eluate is then stored above ground, again cleaning the groundwater, but not degrading the PFAS.

In some cases, the PFAS can be bound by an activated nanocellulose that can be regenerated using an aqueous solution. This contaminated eluate is then stored above ground, again cleaning the groundwater, but not degrading the PFAS.

In some cases, the PFAS can be removed by thermal treatment, and converted to a volatile compound. This vapor can be collected, cooled and condensed. This contaminated condensate is then stored above ground, again cleaning the groundwater, but not degrading the PFAS.

Few options exist to degrade the PFAS. Incineration at high temperatures claims to degrade PFAS, but residues of the PFAS molecules have been found outside the incinerator stack suggesting that these compounds designed to be fire resistant are also resistant to degradation by incineration.

Among the pollutants and toxic compounds is also 1,4-dioxane. 1,4-dioxane (also known as dioxane, p-dioxane, diethylene dioxide, diethylene oxide, diethylene ether and glycol ethylene ether) has been found in groundwater sites throughout the U.S. and has been classified by the EPA as likely to be carcinogenic to humans by all routes to exposure. By 2016, 1,4-dioxane was identified at multiple sites on the EPA's national priority list, also known as superfund sites. Occupational exposure limits and health based drinking water guidance values have been established by federal and state authorities. Exposure can occur through ingestion of contaminated food and water, dermal contact, or inhalation of vapors. No federal maximum contaminant level has been set for drinking water, but the EPA has calculated a one day, a ten day and a lifetime health advisory level for drinking water. The EPA has also published residential and industrial soil screening levels in addition to a residential air screening level.

1,4-dioxane was widely used as a stabilizer in certain chlorinated solvents such as 1,1,1-trichloethane (TCA), and can be found at certain solvent release sites in addition to sites with TCA contamination. 1,4-dioxane is also used as a purifying agent in the manufacture of pharmaceuticals. 1,4-dioxane is a by-product present in some consumer goods such as shampoos, deodorants and cosmetics, and industrial chemicals such as dyes, greases, antifreezes and aircraft de-icing fluids. In addition, 1,4-dioxane is a by-product in the manufacturing of polyethylene terephthalate (PET) plastic and is typically found at PET manufacturing facilities.

1,4-dioxane is fully miscible with water, can leach readily from soil to groundwater and can migrate rapidly in groundwater. Ranking among the most miscible organic contaminants in the saturated zone, 1,4-dioxane can be found farther downgradient than the leading edge of a solvent plume. The presence of 1,4-dioxane has been documented at twice the length of associated solvent plume. 1,4-dioxane's low affinity for organic material in soil accompanied by its low potential to partition from dissolved phase to vapor phase promote its migration to and within groundwater. These factors combined with the findings that 1,4-dioxane is resistant to biodegradation in the subsurface contribute to large dioxane groundwater plumes. Examples include the Gelman plume spreading from the Gelman Sciences Plant and approaching the Huron River where it could threaten Ann Arbor's water supply. The large 1,4-dioxane groundwater plume originating from the Northrop Grumman site in Bethpage in Long Island now measures 4.3 miles long, 2.1 miles wide and up to 900 feet deep, and threatens the local water supply.

Groundwater soils contaminated with 1,4 dioxane are typically treated with advanced oxidation treatment in combination with granular activated carbon adsorption (GAC). Advanced oxidation treatment involves treating the water with hydrogen peroxide and ultraviolet (UV) light or hydrogen peroxide and ozone. Oxidation can also be performed using peroxone and iron activated persulfate oxidation. Oxidation treatments can be performed ex situ where the water is pumped through a reactor system where the resulting by-products can be removed by GAC treatment and the treated effluent can be reinjected. This approach can also be done in situ where the peroxide is injected into the groundwater which is sparged with air. The resulting by-products can be cleaned up by bioremediation. Electrical resistance heating, and phytoremediation and bioremediation have also been used to degrade 1,4-dioxane.

Aerobic microbial degradation of 1,4-dioxane has been described in at least four bacterial species, Amycolata sp. Strain CB1190, Mycobacterium vaccae, and two strains of Rhodococcus sp. are capable of using dioxane as the primary growth substrate. Evidence for the biodegradation of dioxane is from aerobic systems. Additional microbial strains have been shown to catalyze co-metabolic degradation of 1,4-dioxane where other metabolites such as butane or tetrahydofuran (THF) must be present for degradation to occur. Indigenous microbial communities have been used for the co-metabolic degradation of 1,4-dioxane and THF at the Lowry Landfill Superfund site (DiGuiseppi et al., Remediation J., 27(1):71-92, 2016).

The level of success of bioremediation efforts is largely dependent on the type of microbes used and their capability to destroy or degrade toxic compounds. However, current bioremediation approaches have a limited range of treatable pollutants. Furthermore, the degradation products themselves can be toxic and require additional degradation. Other limitations of bioremediation are (i) the complex scientific knowledge required to customize an appropriate treatment plan, (ii) the difficulty of maintaining aerobic conditions to optimize degradation for those strains that require oxygen and (iii) the bioavailability of contaminants.

There is a continuing need for improved methods for remediating soils, sediments, water, and sorbent materials containing PFAS substances, especially those that degrade emerging contaminants such as PFAS and 1,4-diooxane.

### SUMMARY

The present inventors have identified Geobacillus midousuji bacteria that can decontaminate groundwater, soil and sediments or other matrixes (such as sorbent materials) contaminated with 1,4-dioxane and/or PFAS and/or polycyclic aromatic hydrocarbons (PAH) and more specifically, benzo[a]pyrene (BaP), and degrade the 1,4-dioxane and/or PFAS and/or PAHs such as benzo[a]pyrene (BaP).

The present disclosure provides for a method for bioremediation of soil, sediment, wastewater (e.g., groundwater), or sorbent material containing 1,4-dioxane and/or PFAS and/or benzo[a]pyrene (BaP). The method may comprise the steps of: (a) administering Geobacillus midousuji, or a mutant derived therefrom (e.g., having degrading activity of the Geobacillus midousuji), to the soil, sediment, wastewater, or sorbent material to be bioremediated; and, (b) incubating the Geobacillus midousuji in the soil, sediment or wastewater at a temperature ranging from about 40 to about 70° C. and a humidity ranging from about 80% to about 100% for a period of time ranging from about 1 hour to about 20 days.

The Geobacillus midousuji may be strain SH2B (American Type Culture Collection (ATCC) No. 55926) or a mutant derived therefrom (e.g., retaining the degrading activity thereof), strain SH2A (ATCC No. 202050) or a mutant derived therefrom (e.g., retaining the degrading activity thereof), or mixtures thereof.

In certain embodiments, the total PFAS concentration in the soil, sediment, wastewater, or sorbent material after incubation is less than about 100 parts per million (PPM), less than about 50 PPM, or less than about 20 PPM.

In certain embodiments, the 1,4-dioxane concentration in the soil, sediment, wastewater, or sorbent material after incubation is less than about 100 parts per million (PPM), less than about 50 PPM, or less than about 20 PPM.

In certain embodiments, the total BaP concentration in the soil, sediment, wastewater, or sorbent material after incubation is less than about 100 parts per million (PPM), less than about 50 PPM, or less than about 20 PPM.

The Geobacillus midousuji to the soil, sediment, wastewater, or sorbent material ratio may range from about 0.01% to about 1% w/w, from about 0.01% to about 0.1% w/w, from about 0.08% w/w to about 0.15% w/w, or about 0.1% w/w.

In certain embodiments, the Geobacillus midousuji is activated into log phase growth before incubation with the soil, sediment, wastewater, or sorbent material by incubation of the Geobacillus midousuji under an aerobic condition at a temperature ranging from about 60° to about 65° C. in a medium comprising B-complex vitamin and amino-N with trace metals.

The method may further comprise the step of rotating the soil, sediment, wastewater, or sorbent material at least about 1 to about 5 times per day during incubation with the Geobacillus midousuji.

In one embodiment, the Geobacillus midousuji, or a mutant derived therefrom, is administered in a stationary phase, in spore form, in planktonic form, in biofilm form, or a combination of planktonic and biofilm form.

In another embodiment, the Geobacillus midousuji, or a mutant derived therefrom, is administered in the form of a biofilm to the soil, sediment, wastewater, or sorbent material. For instance, the Geobacillus midousuji, or a mutant derived therefrom, may be immobilized in a fixed film bioreactor (e.g., in the form of a biofilm) and then contacted with the soil, sediment, wastewater, or sorbent material. In yet another embodiment, the administered Geobacillus midousuji, or a mutant derived therefrom, is in the form of a biofilm immobilized in a fixed film bioreactor.

In one embodiment, the Geobacillus midousuji in the soil, sediment, wastewater, or sorbent material is inactivated.

The incubation of the Geobacillus midousuji in the soil, sediment, wastewater, or sorbent material may be done under aerobic conditions. The incubation of the Geobacillus midousuji in the soil, sediment or wastewater may be done under anaerobic conditions.

The method may further comprise purifying or harvesting at least one protein product and/or at least one surfactant product.

The present disclosure provides for a kit comprising: (i) Geobacillus midousuji or a mutant derived therefrom; (ii) printed matter with instructions for activating the Geobacillus midousuji into log phase growth before incubation with soil, sediment, wastewater, or sorbent material by incubation of the Geobacillus midousuji under aerobic conditions at a temperature ranging from about 60° to about 65° C. in a suitable growth medium (e.g., such as a growth medium comprising B-complex vitamin and amino-N with trace metals); and, instructions for use of the Geobacillus midousuji in the soil, sediment, wastewater, or sorbent material, wherein the soil, sediment, wastewater, or sorbent material is maintained at a temperature ranging from about 40° C. to about 70° C. and a humidity ranging from about 90% to about 100% for a period of time ranging from about 1 hour to about 20 days.

The Geobacillus midousuji may be strain SH2B (American Type Culture Collection (ATCC) No. 55926) or a mutant derived therefrom (e.g., retaining the degrading activity thereof), strain SH2A (ATCC No. 202050) or a mutant derived therefrom (e.g., retaining the degrading activity thereof), or mixtures thereof.

According to an aspect of the present invention, a method for bioremediation of soil containing 1,4-dioxane and/or PFAS and/or benzo[a]pyrene (BaP) is provided. The method comprises the steps of administering to the soil to be bioremediated, Geobacillus midousuji, designated ATCC55926 strain SH2B, an enhanced bacterial strain of ATCC202050, ATCC55926, or mixtures thereof, at a ratio ranging from about 0.01% to about 1% (w/w of wet weight of the Geobacillus midousuji to weight of the soil). The method also includes the step of incubating the Geobacillus midousuji in the soil, wherein the soil is maintained at a temperature ranging from about 40 to 70° C. and a humidity ranging from about 80% to about 100% for a period of time ranging from about 1 hour to about 480 hours.

According to another aspect, a kit is provided that comprises: Geobacillus midousuji, designated ATCC55926 strain SH2B, an enhanced bacterial strain of ATCC202050, ATCC55926, or mixtures thereof at a ratio ranging from about 0.01% to about 1% (w/w of wet weight of the Geobacillus midousuji to weight of the soil). The kit also includes printed matter with instructions for activating the Geobacillus midousuji into log phase growth before incubation with the soil by incubation of the Geobacillus midousuji under aerobic conditions at a temperature ranging from about 60° to about 65° C. in a suitable growth medium (e.g., a growth medium comprising B-complex vitamin and amino-N with trace metals). In addition, the kit includes instructions for use of the Geobacillus midousuji in the soil for degrading 1,4-dioxane and/or one or more PFAS (such as those described herein) and/or benzo[a]pyrene (BaP), wherein the soil is maintained at a temperature ranging from about 40 to 70° C. and a humidity ranging from about 90% to about 100% for a period of time ranging from about 1 hour to about 180 hours. In another embodiment, the instructions state that the Geobacillus strain can be added at ambient temperature, ranging from about 15° C. to about 35° C.

These and other aspects, features, and advantages can be appreciated from the accompanying description of certain embodiments of the invention and the accompanying drawing figures and claims.

### BRIEF DESCRIPTION OF THE DRAWINGS

FIG. 1 is a flow diagram illustrating an exemplary method for bioremediation of contaminated matter in accordance with one or more embodiments of the invention;

FIG. 2 is a block diagram illustrating a side-view of an exemplary system for bioremediation of contaminated matter according to an embodiment of the present invention;

FIG. 3 is a chart plotting peak height vs. retention time results of degradation experiments with soils containing Benzo[a]pyrene (BaP) and PCBs in accordance with one or more embodiments of the invention;

FIG. 4 is an image illustrating the results of two-dimensional gel electrophoresis of supernatants from a bacterial broth prepared in accordance with one or more embodiments of the invention;

FIG. 5A includes a chart illustrating the results of Matrix-Assisted Laser Desorption/Ionization-Time of Flight analysis of the 54 kDa protein bands prepared in accordance with one or more embodiments of the invention;

FIG. 5B includes a chart illustrating the results of Matrix-Assisted Laser Desorption/Ionization-Time of Flight analysis of the 116 kDa protein bands prepared in accordance with one or more embodiments of the invention;

FIG. 6 is a chart plotting optical density vs. incubation time graphically illustrating the enhancement of strain cell yield by optimizing growth media from Trypticase Soy Broth to a modified Terrific Broth prepared in accordance with one or more embodiments of the invention;

FIG. 7 is a graphical representation of the molecular structure of examples of oxidative biotransformation reactions for monooxygenases and examples of oxidative biotransformation reactions for dioxygenases and perioxiases; and

FIGS. 8A-8C contain photographs of 3 thin-layer chromatography (TLC) plates showing the analysis of extracts of the spent media from the Geobacillus SH2B strain. The plate in FIG. 8A was stained with Phosphomolybdic Acid (PMA) and shows the presence of bands in the samples in lanes 2 and 3 that match the D3 and D4 bands from the di-rhamnolipid standard in lane 1. The plate in FIG. 8B was stained with primuline and imaged under UV light and shows the presence of bands in the samples in lanes 3 and 4 that match the D3 and D4 bands from the di-rhamnolipid standard in lane 2. The plate in FIG. 8C was stained with anthrone and shows the presence of bands in the samples in lanes 3 and 4 that match the D3 and D4 bands from the di-rhamnolipid standard in lane 2.

FIGS. 9A-9C are bar graphs showing the concentration of specific PFAS compounds in parts per billion (ppb) extracted from spent sorbent that had accumulated PFAS from contaminated groundwater. The four PFAS present in the largest concentrations (PFHxS, PFOA, PFOS, and PFPeA) are shown in FIG. 9A, two forms of PFAS present at intermediate concentrations (PFHpS, PFPeS) are shown in FIG. 9B, and eighteen other forms of PFAS present in small concentrations are shown in FIG. 9C, 48 or 72 hours after treatment with a control or Geobacillus miduosuji SH2B as described in Example 1.

FIGS. 10A and 10B are bar graphs showing the content of specific PFAS compounds from a spent sorbent material that had accumulated PFAS from contaminated groundwater. The six PFAS present in the largest concentrations (PFHxS, PFOA, PFOS, 6:2DTS, PFHxA and PFPeA) are shown in FIG. 9A, and eighteen forms of PFAS present at lower concentrations are shown in FIG. 9B, after treatment with Geobacillus miduosuji SH2B or without treatment (a control) as described in Example 2.

FIG. 11 is a graph showing the concentrations of Total Organo-Fluorine (TOF) in parts per billion (ppb) in a solution of PFOS that was treated with Geobacillus miduosuji SH2B (Treatment 1, 2 & 3) over 98 hours, with the results from a control with no added bacteria shown as solid circles.

FIG. 12 are bar graphs showing the concentration of 1,4-dioxane in aqueous solutions after 8 hours of treatment with Geobacillus miduosuji SH2B (T1, T2, and T3) or without treatment (a control) as described in Example 5.

FIG. 13 are bar graphs showing the concentration of 1,4-dioxane in aqueous solutions after 28 hours treatment with Geobacillus miduosuji SH2B or without treatment (a control) as described in Example 6.

FIG. 14 is a graph of the optical density (OD) measured at 600 nm of the treated and control samples in Example 6 during the 28 hour incubation period.

FIG. 15 is a photograph of a biofilm formed by Geobacillus miduosujii as described in Example 7.

### DETAILED DESCRIPTION

The present application incorporates by reference International Publication No. WO 2020/214890 and its priority application U.S. Patent Application No. 62/835,148, filed Apr. 17, 2019.

By way of overview and introduction, systems and methods of the present invention are directed to performing bioremediation using a Geobacillus sp. bacterial strain, for example, Geobacillus midousuji bacterial strain SH2B (ATCC Accession No. 55926) and/or strain SH2A (ATCC No. 202050).

Geobacillus midousuji bacteria produce a variety of chemically and biologically active molecules that can be used to replace harsh reactants in chemical synthesis, green chemistry or bioremediation applications. These compounds include surfactants and proteases, as well as other enzymes that can be used for enantiospecific reactions or the suppression of reactive oxygen species in chemical or biological systems. The bacteria can be used in remediation of chemical waste, including poly-chlorinated biphenyls (PCBs), poly-aromatic hydrocarbons (PAHs), and perfluorochemicals (e.g., perfluorooctanoic acid or PFOA). Reactions can be carried out under either aerobic or anaerobic conditions across a wide range of temperatures, which has the potential to significantly reduce the cost of current remediation.

According to one or more embodiments, various strains of Geobacillus midousuji bacteria can be used to decontaminate soil and sediments or other contaminated matrixes, e.g., water supplies or sorbent materials, of 1,4-dioxane and/or PFAS as well as polycyclic aromatic hydrocarbons (PAH) and more specifically, benzo[a]pyrene (BaP). Because decontamination can occur under either aerobic or anaerobic conditions, the costs of remediation are lower. The embodiments disclosed herein also expand the range of pollutants treatable by bioremediation with Geobacillus midousuji. The disclosed embodiments achieve various beneficial results including, inter alia: extending the applications of Geobacillus midousuji to enantiospecific reactions, surfactants and proteases, and the suppression of reactive oxygen species in chemical and biological systems; effectively remediating chemical waste, including 1,4-dioxane and/or PFAS, polychlorinated biphenyls, poly-aromatic hydrocarbons, including benzo[a]pyrene (BaP), and perfluorochemicals; effectively remediating chemical waste in either aerobic or anaerobic conditions across a range of temperatures (40-70° C.); being usable in packed-bed columns to remediate the aqueous phase of contaminants in groundwater; and reducing remediation costs.

In the present method, the bacteria Geobacillus midousuji may be incubated with the soil, sediment, waste (e.g., wastewater), or sorbent material under oxygen or nitrate (NO₃). The sorbent material can be a material used to bind 1,4-dioxane and/or PFAS and/or benzo[a]pyrene (BaP), for example, from an aqueous solution. For example, the sorbent material can be an ion exchange resin or a cellulose material (e.g., an activated nanocellulose).

PFAS compounds which can be degraded by the method of the present invention include, but are not limited to, carboxylic, sulfonic, and carbonic acids such as perfluorohexane sulfonic acid (PFHxS), perfluorooctanoic acid (PFOA), perfluorooctane sulfonic acid (PFOS), perfluoropentanoic acid (PFPeA), perfluoroheptane sulfonic acid (PFHpS), perfluoropentane sulfonic acid (PFPeS), perfluorobutanoic acid (PFBA), perfluorobutane sulfonic acid (PFBS), perfluorodecanoic acid (PFDA), perfluorododecanoic acid (PFDoA), perfluorodecane sulfonic acid (PFDS), perfluoroheptanoic acid (PFHpA), perfluorohexanoic acid (PFHxA), perfluorononanoic acid (PFNA), perfluorononane sulfonic acid (PFNS), perfluorotetradecanoic acid (PFTA or PFTeDA), perfluorotridecanoic acid (PFTrDA), and perfluoroundecanoic acid (PFUnA); fluorotelomers such as 4:2 fluorotelomer sulfonic acid (4:2 FTS), 6:2 fluorotelomer sulfonic acid (6:2 FTS), and 8:2 fluorotelomer sulfonic acid (8:2 FTS); sulfonamides such as perfluorooctane sulfonamide (FOSA); precursor compounds such as N-ethyl perfluorooctane sulfonamidoacetic acid (NEtFOSAA), and N-methyl perfluorooctane sulfonamidoacetic acid (NMeFOSAA); and replacement compounds such as hexafluoropropyleneoxide dimer acid (HFPO-DA-GenX). In one embodiment, the soil, sediment, waste (e.g., wastewater), or sorbent material includes one or more of the aforementioned PFAS compounds. In another embodiment, the soil, sediment, waste (e.g., wastewater), or sorbent material includes one or more PFAS compounds selected from perfluorohexane sulfonic acid (PFHxS), perfluorooctanoic acid (PFOA), perfluorooctane sulfonic acid (PFOS), perfluoropentanoic acid (PFPeA), perfluoroheptane sulfonic acid (PFHpS), perfluoropentane sulfonic acid (PFPeS), 6:2 fluorotelomer sulfonic acid (6:2 FTS), perfluorobutanoic acid (PFBA), perfluorodecanoic acid (PFDA), perfluoroheptanoic acid (PFHpA), and perfluorohexanoic acid (PFHxA).

Polycyclic aromatic hydrocarbons (PAH) are a class of environmental pollutants that include carcinogenic forms. They are produced by the incomplete combustion of organic material and in particular, the combustion of petroleum products.

Among the PAHs, benzo[a]pyrene (BaP) is a potent carcinogen and is found in high concentrations in cigarette smoke, processed meat, as well as polluted water, soil, and air. https://pubchem.ncbi.nlm.nih.gov/compound/benzo-a-pyrene, retrieved 12/5/18. The systems and methods disclosed herein, which use these bacterial strains, are capable of effectively degrading BaP, one of the more toxic PAH pollutants. Furthermore, the bacteria used herein are capable of efficiently degrading other ring-based organic contaminants that are also harmful to human health. PAHs are a large group of organic compounds with two or more fused aromatic rings. They have a relatively low solubility in water but are highly lipophilic. Eight PAHs (Car-PAHs) typically considered as possible carcinogens are: benzo(a)anthracene, chrysene, benzo(b) fluoranthene, benzo(k)fluoranthene, benzo(a)pyrene (BaP), dibenzo(a,h)anthracene, indeno(1,2,3-cd)pyrene and benzo (g,h,i) perylene. The US Environmental Protection Agency (EPA) has promulgated 16 unsubstituted PAHs (EPA-PAH) as priority pollutants. (Srogi, Monitoring of environmental exposure to polycyclic hydrocarbons: a review, Environ Chem Lett 5:169-195 (2007)).

Furthermore, in accordance with one or more of the embodiments, the bacteria described herein can be used to treat: PCBs, dioxins and chlorinated compounds in soil and sediments; dioxane in groundwater. Furthermore in accordance with one or more of the embodiments, the bacteria is capable of efficiently remediating a growing range of chlorinated solvents and other recalcitrant compounds including: dioxins and furans (with tests showing reduction in dioxin levels by 60% in 6 days based on shake flasks and bench scale experiments); PCBs (with tests showing reduction in PCB levels by 50% in 6 days based on lab and scale up experiments); PAHs (with tests showing rapid reduction of PAHs including BaP reduced by 95% in 48 hrs.); BTEX (with tests showing decrease in the concentration of the BTEX components (Benzene, Toluene, Ethylbenzene and Xylene)); and chlorinated solvents (RCRA-US Federal Resource Conservation and Recovery Act, https://www.epa.gov/rcra, retrieved Apr. 15, 2020).

Geobacillus midousuji (e.g., strain SH2B, ATCC No. 55926; and/or strain SH2A, ATCC No. 202050) are capable of effectively degrading PFAS and BaP in an environmental situation with contaminated matter such as soil, sediments, water (e.g., groundwater and wastewater), and other contaminated matrixes, such as sorbent materials. The term “bacteria” is used throughout the text to refer to Geobacillus midousuji, as well as more generally, any other bacterial species that are found to have significant contaminant degrading capabilities. Examples of Geobacillus midousuji include strain SH2B (ATCC Accession No. 55926), and/or strain SH2A (ATCC Accession No. 202050). Embodiments of the invention can provide both an aerobic and anaerobic solution for treating pollutants through bioremediation. The methods and systems provided herein can reduce remediation costs, reduce energy costs, expand the breadth of pollutant targets, offer a cell-free degradation system, and can be used in a kit.

To the extent certain exemplary embodiments and tests are shown and described as being based mainly on specific strains derived from Geobacillus midousuji, for instance, ATCC Accession No. 55926 strain SH2B, the invention is not necessarily so limited. For example, beneficial results are suggested for configurations using ATCC Accession No. 202050 strain SH2A. Discussion of Geobacillus midousuji and exemplary approaches for degrading matter with microorganisms ATCC 55926 or 202050 are shown and described in U.S. Pat. Nos. 6,420,165 and 6,190,903. The Geobacillus strains referred to herein deposit details are: (i) bacterium strain, Bacillus midousuji SH2A deposited on Jan. 21, 1997 with the American Type Culture Collection (ATCC), 12301 Parklawn Drive, Rockville, Md. 20852, U.S.A., under the provisions of the Budapest Treaty for The International Recognition Of The Deposit Of Microorganisms For The Purposes Of Patent Procedure. Bacterium strain SH2A has been accorded ATCC Accession Number 55926; and, (ii) bacterium strain, Bacillus midousuji SH2B deposited on Oct. 24, 1997 which the American Type Culture Collection (ATCC), 12301 Parklawn Drive, Rockville, Md. 20852, U.S. A., under the provisions of the Budapest Treaty for The International Recognition of The Deposit of Microorganisms For The Purposes of Patent Procedure. Bacterium strain SH2B has been accorded ATCC Accession No. 202050.

The organic material degraded by the bacterial strains above include, but should not be limited to, plastics, specifically polyethylene. In one specific embodiment, the polyethylene may be irradiated prior to treatment with the bacterial strains to facilitate the degradative process. Specifically, in such an embodiment, the polyethylene may be irradiated with ultra-violet light.

The organic material degraded by the bacterial strains above may also comprise a protein, specifically waste products of households and such industries as food-processing, agriculture, dairy or fisheries. Specific examples include, but are not limited to, wood pulp, paper products, shellfish, coffee bean dregs, tuna-fish heads, squids and other by-products of these industries. Further kitchen waste may also be degraded by these bacteria. Kitchen waste may include, but are not limited to, paper products, shellfish, coffee bean dregs, tuna-fish heads, squids and other by-products found in kitchen bins, trash dumps and other consumer-based waste.

Organic materials comprising a sugar can be degraded by the methods described above. Specifically, such sugars include, but are not limited to, mannose, maltose, trehalose, fructose and raffinose. Many of these sugars may be found in kitchen wastes and are by-products of industries in food-processing and agriculture, e.g. fruits.

The organic material also includes amino acid-based compounds. These compounds may be proteins, polypeptides, peptides, naturally occurring or synthetic. The organic material may also include a nucleic acid molecule, specifically deoxyribonucleic acid molecules. DNAase activity has been described in these bacterial strains. U.S. Pat. No. 6,420,165.

The present methods and compositions may be used to treat a waste. The waste may be an industrial waste, an agricultural waste, a human municipal waste, fertilizers, domestic sewage, and industrial effluents. The waste may be manufactured gas plant waste, soot, or used petroleum products. The waste may be produced from incomplete combustion of organic matter.

The present methods may produce at least one protein product which may be chemically and/or biologically active. such as enzymes, protease, 1-pyrroline-carboxylate dehydrogenase (P5C), aconitate hydratase (aconitase), a dioxygenase. The protein product(s) may be used in an enantiospecific reaction. The protein product(s) may be an enantiospecific enzyme, a surfactant, and/or an agent for suppressing reactive oxygen species in a chemical and/or biological system.

### Exemplary Bioremediation Method:

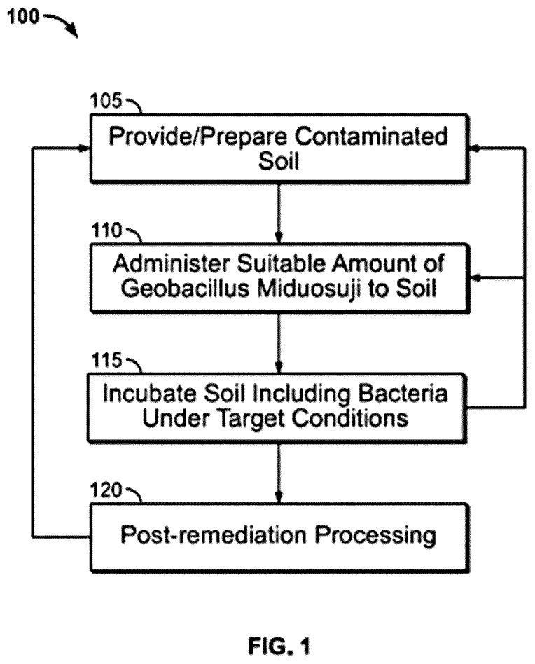

FIG. 1 is a flow diagram illustrating an exemplary method 100 for bioremediation of contaminated matter in accordance with one or more embodiments of the invention. Steps of the method 100 can be performed in situ, i.e., without removing the material from its original position, or ex-situ, after removal of the soil, sediment, water, etc., from its original location or site, or a combination of either of the foregoing.

In-situ bioremediation involves remediation of soils in the ground without removal of the soil to a different location. The exemplary method 100 can also be performed ex-situ, for instance, near the original location of the contaminated material, or at a designated soil remediation site some distance away. Ex-situ bioremediation is a biological process in which excavated soil is placed in a lined above-ground treatment area. The development of the microbial culture within the remediation zone can be in the presence of oxygen, i.e., under aerobic conditions, or without oxygen, i.e., under anaerobic conditions. The incubation conditions can be dependent on the nature of the contaminants that require degradation and their environmental context. As noted, Geobacillus midousuji ATCC 55926 strain SH2B can provide for both an aerobic or anaerobic solution for treating pollutants, including, but not limited to 1,4-dioxane, PFAS, and BaP. The preferred embodiment for most contaminants is to carry out the microbial incubation under aerobic conditions because the rates of contaminant degradation are typically greater.

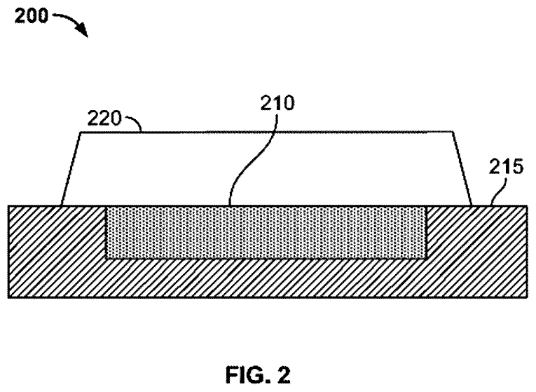

At step 105, a volume or weight of the contaminated soil requiring remediation is provided. For the purpose of illustration, method 100 is described in the context of remediating soil contaminated with 1,4-dioxane and/or PFAS and optionally BaP in situ. FIG. 2 further illustrates the exemplary system 200 in which the exemplary bioremediation method 100 is implemented in accordance with one or more embodiments of the invention. Specifically, FIG. 2 is a side-view of the system 200 showing the cross section of the volume of soil 210 and the surrounding earth 215.

Step 105 can include measuring certain properties of the soil volume to determine baseline soil properties which can be used to inform subsequent steps of the remediation process, as further described herein. The measurements can be collected using measuring devices (not shown) and techniques that are known in the art, including, for example, the measurement of pH, oxidation-reduction potential (ORP), and the contaminant and biological environment using thin layer chromatography (TLC), high pressure liquid chromatography (HPLC), gas chromatography (GC), GC-mass spectroscopy (GC-MS), liquid chromatography—mass spectrometry (LC-MS/MS), Time of flight Ion Mass Spectrometry (TOF-SIMS), matrix assisted, laser desorption/ionization-time of flight Mass Spectrometry (MALDI-ToF MS), gene amplification and genetic sequencing for genomics and metagenomics, next-gen sequencing of cDNA for transcriptomics, quantitative polymerase chain reaction (qPCR) and other methods. https://www.atsdr.cdc.gov/toxprofiles/tp69-c6.pdf, retrieved Oct. 20, 2021. Preferably, measurements taken from the soil include the concentration of at least 1,4-dioxane, PFAS, BaP, or other contaminants of interest. Other measured parameters can include moisture content, the size distribution of the soil, temperature of the soil and aeration level. It should be understood that one or more of the properties can be measured prior to, during or after remediation. Other measurements relating to the presence of contaminants in the environment, e.g., sediments, water, air, can also be taken in a similar manner as that performed with soil.

Step 105 can further include steps for preparing the soil 210 for remediation. Soil preparation can be performed using devices and techniques that are known in the art. The soil preparation can be performed as a function of the measured soil properties to, for instance, ensure that the soil properties are suitable for effective bioremediation of the soil. For instance, soil or sediment preparation can include screening the material to remove rocks and debris larger than a few inches, watering or drying the soils to provide optimum moisture conditions and adjusting the pH of the soil with acids or bases.

At step 110, the bioremediation bacteria Geobacillus midousuji is administered to the soil. More particularly, in one embodiment, ATCC Accession No. 55926 strain SH2B can be administered using techniques that are known in the art, e.g., spraying or mixing the bacteria with batches of the soil. Preferably an effective, degrading amount of the bacteria is administered to the soil. For example, the bacteria to soil ratio can range from about 0.001% to about 1% (w/w of wet weight of the Geobacillus midousuji to weight of the soil) and more preferably from about 0.01% to about 0.1% (w/w). Other ranges, include, 0.05-0.5% (w/w), 0.02%-0.6% and 1%-5%.

The bacteria can be administered in an amount that is suitable for degrading the amount of 1,4-dioxane and/or PFAS and optionally BaP in the soil to an acceptable level as mandated by a government regulatory body. For example, the U.S. Environmental Protection Agency (EPA) defines regional screening level (RSL) for BaP in residential and industrial soil. In its Integrated Risk Information System, the U.S. EPA has published updated toxicity values for BaP stating that, at the time of publication, the updated RSL for residential and industrial soil, respectively, is 0.11 mg/kg (ppm) and 2.1 mg/kg (ppm) https://www.epa.gov/iris, retrieved Oct. 20, 2021. By way of further example, using the EPA IRIS site (https://www.epa.gov/risk/regional-screening-levels-rsls-generic-tables, accessed Oct. 20, 2021), the table identifies, in the “Cancer” column for “residential” and “Composite worker soil,” SLs of 1.8 mg/kg (ppm) and 22 mg/kg (ppm), respectively, for these populations using a THQ of 0.1. Using the RSL model, the RSL is 1.78 mg/kg (ppm) for the residential soil and 66 mg/kg (ppm) for the construction soil. U.S. EPA defined RSLs for PFAS have not yet been determined, although a growing number of individual states have established regulatory limits from 0.027 to 300 parts per trillion (ppt) for PFOS and PFOA in drinking water. Various European agencies are adopting similar guidelines. The U.S. EPA defined RSL for 1,4-dioxane in groundwater is 94 parts per trillion (ppt).

Accordingly, the amount of bacteria administered can be a function of the level of 1,4-dioxane and/or PFAS and optionally BaP in the soil as well as a target level after remediation, e.g., more bacteria would be used for soils with 10 mg/kg (PFASor BaP weight/soil weight) than for soils with 2.1 mg/kg (PFAS or BaP weight/soil weight). The amount of bacteria administered can also vary depending on the presence of other soil contaminant(s), the particular microbial culture(s) used to degrade said contaminant(s), the particular bioremediation approach, as well as other properties of the soil. In one embodiment, the amount of bacteria to be administered to the soil can range from about 0.01%, weight of the Geobacillus midousuji to weight of the soil, to more than 1%. The initial inoculate of bacteria also can be increased to accommodate greater concentrations of 1,4-dioxane and/or PFAS and/or BaP in the soil or sediments. The preparation of the bacteria for soil treatments also can be varied. The bacteria may first be brought into logarithmic growth phase prior to its inoculation to the soil, or the bacteria can be applied to the soil while it is still in a dormant stage from storage.

In view of the present disclosure, determination of “an effective, degrading amount” as described herein is within the knowledge of one skilled in the art and various other methods can be used to determine the amounts of the bacteria required to effectively degrade the waste of interest. In the case of 1,4-dioxane and/or PFAS and/or BaP and Geobacillus midousuji, the amount of 1,4-dioxane and/or PFAS and/or BaP can be determined using any technique known in the art, e.g., thin layer chromatography (TLC), high pressure liquid chromatography, gas chromatography (GC), GC-mass spectroscopy (GC-Mass Spec), Time of Flight Ion Mass Spectrometry (TOF-SIMS). The terms “effective” or “effective, degrading amount” can, in certain embodiments, refer to standard levels set by a governmental agency, e.g., US EPA.

Then, at step 115, the volume of soil to which the bioremediation bacteria have been added is incubated. Incubation of the bacteria, which facilitates the breakdown of 1,4-dioxane and/or PFAS and/or BaP, can require certain environmental conditions. For example, it can be preferable to incubate Geobacillus midousuji bacterial strain ATCC Accession No. 55926 strain SH2B and/or ATCC 202050 strain SH2A at 40° to 70° C. Incubation and thus, the breakdown of 1,4-dioxane and/or PFAS and/or BaP can be facilitated by maintaining preferable incubation conditions. Accordingly, step 115 can include at least partially enclosing the treatment area using, for example, a plastic liner or a temporary greenhouse-type enclosure 220, as shown in FIG. 2. Step 115 can also include controlling the ambient temperature within the enclosure such that the target temperature of the soil is maintained during the incubation period. Step 115 might also include the supply of air and/or oxygen and moisture to the environment within the enclosure 220 and, more generally, within and around the soil 210 using approaches that are known in the art and, as such, any such techniques can be used in connection with method 200.

Soil preparation can vary depending on the particular bioremediation approach, e.g., aerobic or anaerobic, low or high pH. Soil preparation can also vary depending on the type of soil contaminant(s) or the type of bacterial culture(s) used to degrade said contaminant(s).

The incubation can also be performed for an amount of time that is suitable for effective degradation of the contaminants by the applied bacteria. For example, Geobacillus midousuji bacterial strain ATCC Accession No. 55926 strain SH2B and/or ATCC 202050 strain SH2A can be incubated with the soil at a humidity ranging from about 80% to about 100% for a period of time ranging from about 1 hour to about 40 days. The prescribed amount of time can be predetermined based on parameters including the soil conditions, contaminant levels, the amount of bacteria applied, their degradation rates and the final contaminant levels desired and environmental conditions. The duration of the incubation can also be defined as a function of the estimated degradation rate for the contaminant(s) in question and confirmed by subsequent measurements of the remaining concentration of contaminants, e.g., 1,4-dioxane and/or PFAS and/or BaP.

It should be further understood that one or more aspects of steps 105-115 can be repeated during incubation to effect further removal of the contaminants. Also, during incubation, aeration may be accomplished by periodically rotating the soil volume using techniques known in the art. In one embodiment, rotating can be performed once a day and can involve mixing the soil using an auger, a garden-tiller, a backhoe, shovel or other such soil processing equipment known in the art. Rotation also can be accomplished continually by incubating the material in a rotating chamber.

Incubation of the soil can require watering the soil regularly, in such a manner that the water content of the soil remains at an optimum level and therefore, degradation rate of the contaminants maintains at a maximum rate.

Furthermore, during incubation, the concentration of the bacteria and/or the soil contaminants and/or other measurable conditions of the soil or environment can be measured and monitored at least periodically, e.g., every 1, 2, 3, 4, 5, 10, 20, 30, 40, 50, 100 or 180 hours or more frequently as is needed. More preferably, the conditions can be monitored every 12, 24, 48, 96, or 192 hours. Thus, dynamic adjustments can be made during incubation based on the monitored conditions so as to facilitate the breakdown of the contaminants by the bacteria. For instance, additional amounts of the bacteria can be re-administered and distributed within the soil volume to adjust for process-loss and the pH of the soil can be maintaining in an optimum range.

The steps of the bioremediation method 200 can be implemented using a control system. The control system can be configured to actively monitor and dynamically control the operation of the bioremediation system 200 to improve operation and efficiency. For example, one or more of the aforementioned operational input parameters of the system and real-time conditions can be measured, monitored and analyzed using the control system before and/or during operation. Accordingly, the control system can define or adjust certain controllable system parameters so as to optimize the efficiency of the bioremediation system.

In addition, as shown in FIG. 1, the method 100 can further include, step 120, in which the soil is treated following incubation. Post-incubation treatment can be performed to enhance the degradation of the contaminants by the microbial population. Post-incubation treatment can also be performed to enhance degradation of any by-products of the bioremediation process. Post-incubation treatment such as heating above 100° C. also can be used to destroy the remaining bacteria that were introduced initially.

In addition to the application of the Geobacillus strains to soil decontamination, this bioremediation is applicable to the decontamination of water and groundwater in which the contaminants are in dissolved, aqueous form. In this application, the bacteria and their associated enzymes are used to break-down water-miscible contaminants such as volatile organic compounds (chloroform, bromodichloromethane, dibromochloromethane) solvents, perchloroethene (PCE), trichloroethene (TCE), 1,1,1-trichloroethane (TCA), chloromethane and methylene chloride) gasoline hydrocarbons such as benzene, toluene, ethylbenzene and xylene (BTEX) and oxygenates and refrigerants. The Geobacillus treatment also is applicable to emerging contaminants in ground water such as 1,4-dioxane and PFAS.

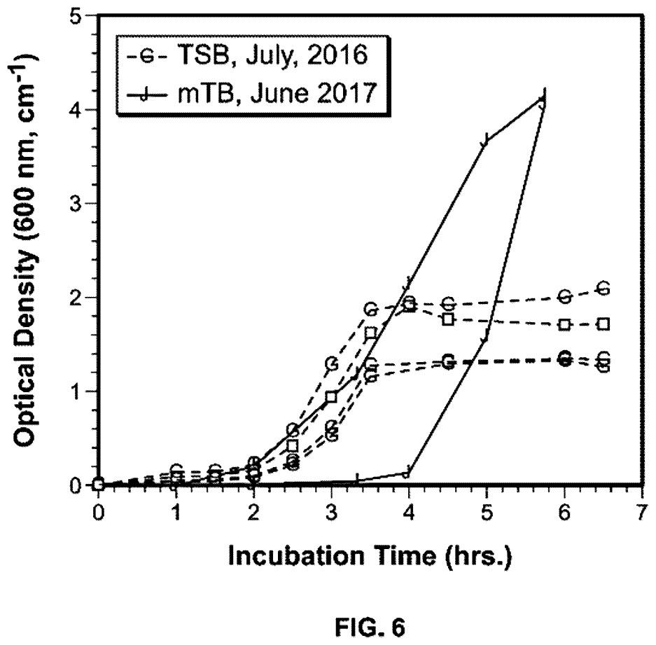

Geobacillus midousuji bacterial strain ATCC Accession No. 55926 strain SH2B and/or ATCC 202050 strain SH2A can be used with the methods of the present invention. In certain embodiments, the Geobacillus strain, e.g., SH2B (ATCC Accession No. 202050) may be modified or enhanced by pre-culture of the bacteria as described below (designated “enhanced bacterial strain”) for improved growth, viability during storage, activity in environmental remediation and the production of useful products as follows. The initial isolate of the current strain used is Geobacillus midousuji bacterial strain ATCC Accession No. 202050 strain SH2B or, Geobacillus midousuji bacterial strain ATCC Accession No. 55926 strain SH2A. Growth enhancement of the Geobacillus midousuji bacterial strains was accomplished by optimizing media and growth conditions. In particular, the strains were found to grow better in a modified version of Terrific Broth (mTB) than in the Trypticase Soy Broth (TSB) that was specified in the original description of the strains. The mTB was further optimized to support the growth of G. midousuji by removing the glycerol component and adding a complement of trace metals to produce a Terrific Broth modified specifically to improve the titer of cells and their oxidases. Specifically, the new media formulation increased total cell yield from <2.0×10⁹ cells/ml to over 4×10⁹ cells/ml, as shown in FIG. 6.

The viability of the cells during storage also was optimized through a series of strain selections in the laboratory. This was evidenced by the decrease in the lag period before the beginning of rapid growth from ˜5 hrs. for unenhanced cells, to less than 3 hrs. for cell lines that were optimized for storage. In this way, the phenotype of the current working strain (SH2B2) has been improved by growth selection for cell production and storage by freezing at −80° C. to be superior to the characteristics of the initial isolate available from ATCC. Table 1 below provides the components and amounts of an exemplary mTerrific Broth with Sucrose.

TABLE 1

| Component | Amount/L | |
|---|---|---|
| Yeast extract | 24 | g |
| Tryptone | 12 | g |
| dipotassium phosphate (K₂HPO₄) | 9.7 | g |
| monopotassium phosphate (KH₂PO₄) | 2.2 | g |
| NaCl | 1 | g |

| | |
|---|---|
| MnCl₂ * 4H₂O | 1 ml of 100 mM solution |
| FeSO₄ * 7H₂O | 1 ml of 1 mM solution |
| CaCl₂ * 2H₂0 | 1 ml of 910 mM solution |
| MgSO₄ * 7H₂O | 1 ml of 590 mM solution |

FIG. 6 is a chart plotting optical density vs. incubation time and graphically illustrates the enhancement of strain cell yield by optimizing growth media from TSB to a modified Terrific Broth. Using the modified Terrific Broth (mTB), we isolated a strain that reaches a cell titer of over 4.0×10⁹ cell/ml and exhibits a lag time for the growth curves of approximately 3 hours.

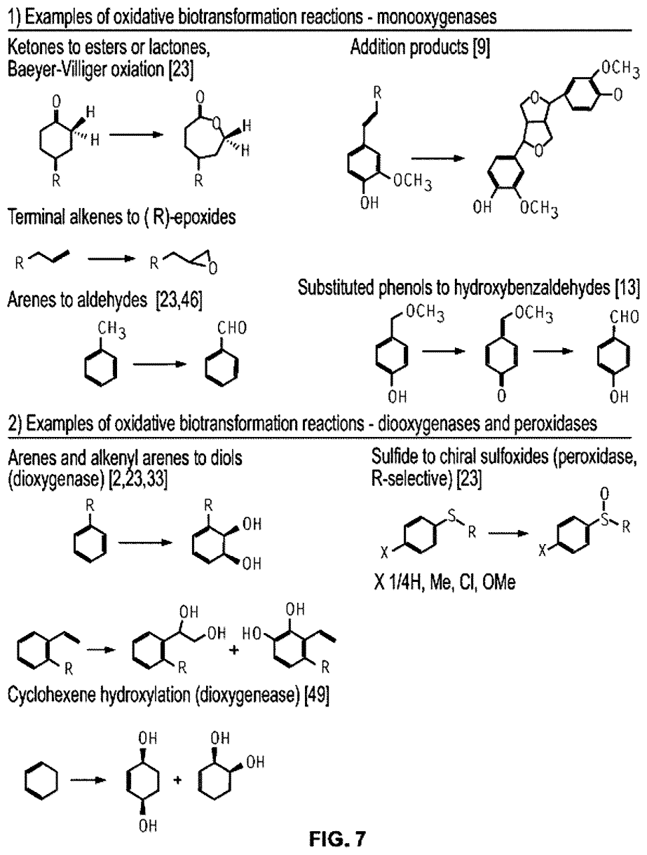

In view of the foregoing it can be appreciated that embodiments of the disclosed invention achieve various benefits including such as expanding the commercial application of strain Geobacillus midousuji ATCC Accession No. 55926 strain SH2B to the degradation of BaP and, 2) the production of intercellular and extracellular proteins. The production of extracellular proteins by Geobacillus midousuji bacterial strain ATCC Accession No. 55926 strain SH2B2 provides a basis for production of useful industrial enzymes for extracellular use. Total extracellular protein production at the end of the logarithmic growth phase of bacterial strain ATCC Accession No. 55926 strain SH2B2 ranged from 0.94 to 1.47 mg/ml, and was composed of over 20 distinct proteins as indicated by gel electrophoresis. These industrial applications include the use of enantiospecific reactions based on the dioxygenase and aconitase enzymes. FIG. 7 illustrates examples of oxidative biotransformation reactions for monooxygenases and examples of oxidative biotransformation reactions for dioxygenases and peroxidases.

The methods and systems of the invention also include kits together with printed material containing instructions on use.

Embodiments of the invention disclosed herein further provide advantages in that they avoid the use of toxic chemicals and reactions involving non-polar solvents.

### BaP Degradation

Testing results demonstrate that the Geobacillus strain described herein is capable of degrading Benzo[a]pyrene (BaP), a potent toxin produced by the incomplete combustion of organic matter (FIG. 1 in supplemental material). This conclusion is based on an incubation of soil to which BaP was added to a concentration of ˜10 ppm and a mixture of polychlorinated biphenyls (PCBs) were added at a concentration of ˜20 ppm. Geobacillus midousuji ATCC Accession No. 55926 strain SH2B was added to 3 separate incubation chambers containing 10 ml of soil. A fourth chamber was prepared as a control containing all of the same ingredients except the bacterium. The soil was incubated at 60° C. for 5 days while being rotated. After 5 days, all of the treated incubations had significantly lower BaP and PCB levels than the control. While the Geobacillus strain previously was shown to degrade dioxins and polychlorinated biphenyls (PCBs), this is the first demonstration of its ability to degrade BaP. In another embodiment, the soil is not incubated at 60° C. for 5 days and the Geobacillus strain is added to the soil which is maintained at ambient temperature, ranging from about 15° C. to about 35° C.

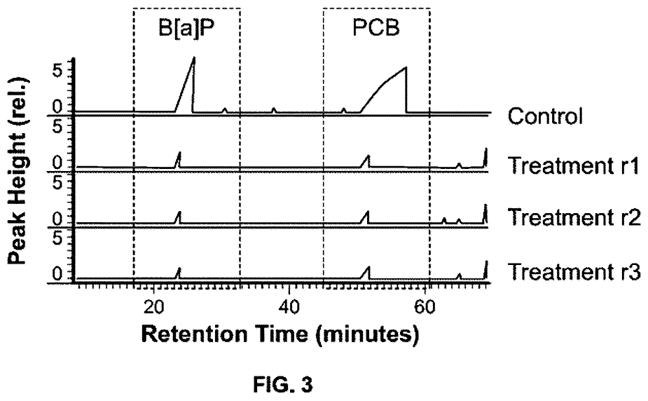

Results of degradation experiment with soils containing Benzo[a]pyrene (BaP) and PCBs are shown in FIG. 3. The graphic shows the gas chromatographic analysis of the extracted contaminants after 5 days of incubation at 62° C. ‘Control’ refers to the incubation without the Geobacillus strain. The three chromatograms marked ‘Treatment’ are triplicate analyses of the same sample incubated with the Geobacillus strain.

### Excreted Protein Products

### A. Number of Peaks

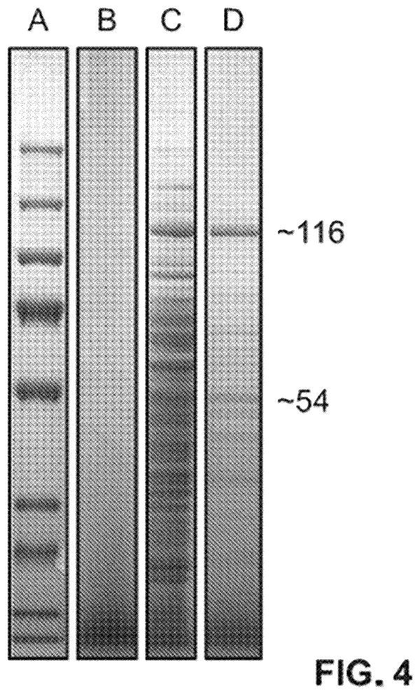

A number of proteins appeared in the supernatant during the growth of the Geobacillus strain. Proteins were sampled in late logarithmic phase after growth on modified Terrific Broth. FIG. 4 illustrates the results of two-dimensional gel electrophoresis of supernatants from the bacterial broth. The leftmost lane ‘A’ shows protein size makers; lane ‘B’ shows fresh medium before growth; lane ‘C’ shows the supernatant from aerobic culture conditions; and lane ‘D’ denotes supernatant from anaerobic culture conditions. These results were generated consistently from samples taken from shake flask experiments as well as from stirred batch bioreactors. In either case, the supernatant from the centrifuged fermentation broth was concentrated with a dialysis membrane to ˜20 times its original concentration. This concentrate was then loaded into the wells of polyacrylamide gels and its proteins separated by electrophoresis. The production of these proteins was significantly greater during aerobic growth than during anaerobic growth, but several proteins maintained significant bands in both growth conditions.

### B. Peak Characterization

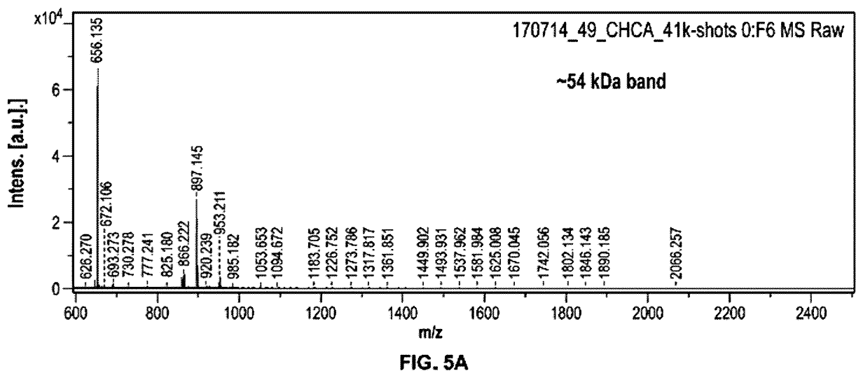

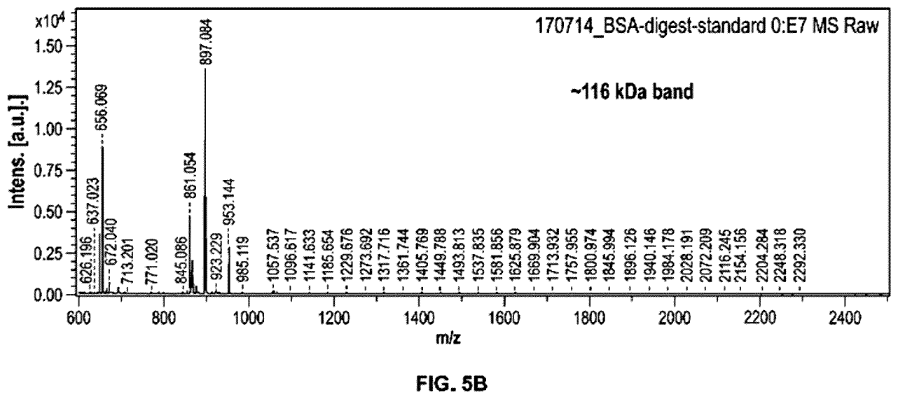

The 54 and 116 kDa protein bands were characterized further because they were the dominant bands that maintained their intensity in both aerobic and anaerobic growth. These bands were digested from the gel after electrophoresis and analyzed by Matrix-Assisted Laser Desorption/Ionization-Time of Flight (MALDI-Tof) mass spectrometer analysis. Peptides that were recorded in both positive and negative beam modes were considered most significant, although some of the other peaks may represent valid peptides as well. Oligonucleotides may also appear on these scans, but matrices such as a-Cyano-4-hydroxycinnamic acid (CHCA) were used to minimize their peaks. FIGS. 5A and 5B illustrates the results of MALDI-Tof analysis of the two proteins. In both cases, samples were prepared with CHCA matrix (saturated) 7:3 water/MeCN with 0.1% TFA and mixed with sample 7:3.

Two bacterial proteins from the current MALDI-ToF database were most similar to these peaks based on the peptide peaks they generated and are shown in Table 2, below. Previously sequenced proteins matching those analyzed from the Geobacillus midousuji bacterial strain ATCC Accession No. 55926 strain SH2B supernatant broth. Protein IDs were based on the closest matches of the peptide peaks detected by MALDI-ToF between the unknown proteins and those in the MALDI-ToF database.

TABLE 2

| Protein ID | Source organism | MW (kDa) |
|---|---|---|
| 1-pyrroline-carboxylate | Geobacillus | 56.5 |
| dehydrogenase | thermodenitrificans | |
| Aconitate hydratase | Bacillus subtilis | 99.3 |

### C. Commercial Use of Excreted Proteins

The amount of enzymes secreted into the growth media and the relative ease of harvesting proteins from this matrix provides a basis for evaluating the commercial applications of this protein pool. The total amount of protein excreted by the Geobacillus strain is 0.94 to 1.47 mg/ml by the end of logarithmic growth. The aconitase peak is estimated to be approximately 10% of the total protein pool and the 1-pyrroline-carboxylate dehydrogenase peak to be approximately 5% of the total pool. On this basis, the production of aconitase and 1-pyrroline-carboxylate dehydrogenase during one of the optimized, batch fermentation runs would be approximately 0.12 and 0.06 mg/ml respectively.

### Dioxygenase Capacity

The capacity of the Geobacillus strain to degrade ring-based organic contaminants has been attributed to its expression of the upper biphenyl pathway. The limiting step in this pathway is the first enzyme, a dioxygenase. The enzyme is denoted as a phenol- or toluene-dioxygenase based on the substrate used for its assay. It has been confirmed that the Geobacillus midousuji ATCC Accession No. 55926 strain SH2B expresses a toluene dioxygenase based on an enzyme specific assay as illustrated in FIG. 6. Specifically, FIG. 6 illustrates the results of an enzyme assay to test for the presence of the toluene dioxygenase enzyme in the Geobacillus strain. The build-up in indoxyl concentration (solid blue circles) indicates the presence of toluene dioxygenase acting on indole as a substrate. The open red squares reflect the subsequent buildup of indigo dye from indoxyl. The major enzyme responsible for the degradation of PCBs appears to be this dioxygenase.

The strains of the bacteria Geobacillus midousuji (G. miduosuji) and having ATCC accession No. 202050 or ATCC No. 55926 make a variety of chemically and biologically active molecules both inter- and extra-cellularly. These discoveries significantly extend the commercial applications of G. midousuji to enantiospecific reactions, surfactants and proteases, and the suppression of reactive oxygen species (ROS) in chemical and biological systems. Additional discoveries extend the types of organic pollutants which the SH2B strain can degrade to include poly-aromatic hydrocarbons (PaHs) such as the highly toxic benzo[a]pyrene, and emerging contaminants such as perfluorochemicals like perfluorooctanoic acid (PFOA) and 1,4-dioxane that currently lack cost effective treatments.

Bioproduct production and pollutant degradation were observed in laboratory culture experiments of several types. In one, the G. midousuji strains were incubated in a variety of liquid growth media including at various times and in various combinations, tryptone, peptone and yeast extract supplemented with various trace metals. Growth rates were optimum at about 62° C., although growth was recorded from 40-70° C., a temperature range that provides flexibility in the design of remediation applications. Growth rates were maximum with aeration, but growth was also confirmed using nitrate (NO₃) instead of oxygen as the electron acceptor.

Bioproducts were analyzed in both the spent media and in cellular material after cell lysis by sonication. Cultures produced significant surfactants as evidenced by foaming of the media and the lowering of liquid surface tension. Proteinaceous material was analyzed by gel electrophoresis and matrix-assisted laser desorption/ionization—time of flight (MALDI-ToF) mass spectrometry of individual protein bands. Several proteins were unique to aerobic growth and have applications in the suppression of ROS in chemical reaction systems and the bio-medical field. The enzyme toluene 1,2 dioxygenase was demonstrated in the intercellular products by an enzyme assay. This Rieske non-heme iron oxygenase can be extracted and used in a cell-free mode for the degradation of aromatic ring structures in contaminant remediation and to carry out enantiospecific reactions for applications in green chemistry.

Other tests demonstrated that G. midousuji could degrade PCBs anaerobically. Growth conditions were as follows. Approximately 30 kg of soil contaminated with PCBs was mixed with G. midousuji and a solution of trypticase soy broth and 500 mM sodium nitrate and the mixture heated to 60° C. for several weeks. This produced a significant decrease in PCBs and indicates that destruction of pollutants is possible without aerating the material, significantly reducing remediation costs.

Several successful incubation approaches were used. These included shake flask experiments with 150-ml and 300-ml liquid culture, stirred batch reactors with and without oxygen, 28-L fermentation reactors and packed-bed columns with live cells and glass beads. The packed-bed columns provide a way to remediation aqueous phase contaminants such as 1,4-dioxane in groundwater.

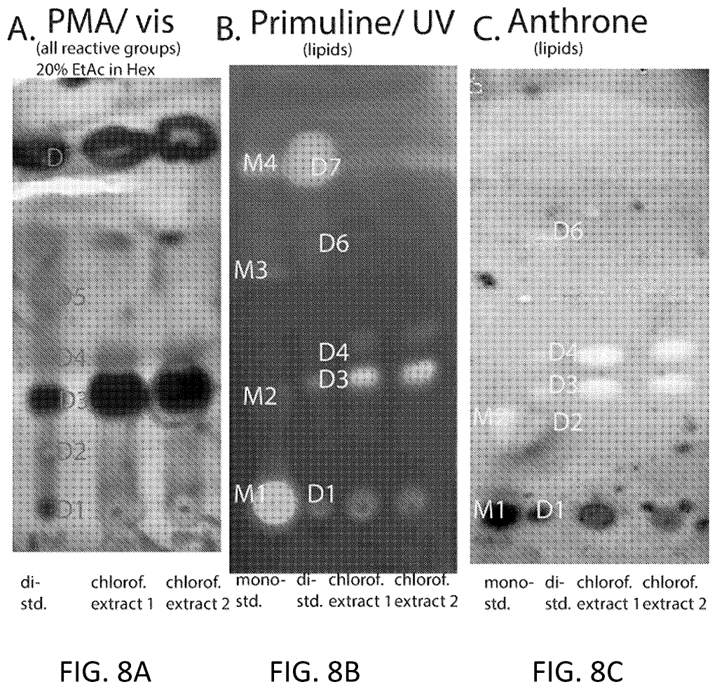

FIG. 8 contains photographs of 3 thin-layer chromatography (TLC) plates showing the analysis of extracts of the spent media from the Geobacillus SH2B strain. Plate A was stained with Phosphomolybdic Acid (PMA) and shows the presence of bands in the samples in lanes 2 and 3 that match the D3 and D4 bands from the di-rhamnolipid standard in lane 1. Plate B was stained with primuline and imaged under UV light and shows the presence of bands in the samples in lanes 3 and 4 that match the D3 and D4 bands from the di-rhamnolipid standard in lane 2. Plate C was stained with anthrone and shows the presence of bands in the samples in lanes 3 and 4 that match the D3 and D4 bands from the di-rhamnolipid standard in lane 2. Rhamnolipids are a class of biosurfactants which contain rhamnose as the sugar moiety linked to β-hydroxylated fatty acid chains. Rhamnolipids can be widely applied in many industries including petroleum, food, agriculture and bioremediation etc. Rhamnolipids are efficacious in bioremediation of organic and heavy metal polluted sites. They also facilitate degradation of waste hydrocarbons such as crude oil and vegetable oil.

As can be appreciated, the foregoing simulations and test results illustrate the benefits of the exemplary bioremediation systems and methods in accordance with embodiments of the present invention.

Testing results demonstrate that the Geobacillus strain described herein is capable of degrading 1,4-dioxane and/or PFAS, an emerging groundwater contaminant that the Environmental Protection Agency (EPA) considers a probable human carcinogen.

### Example 1

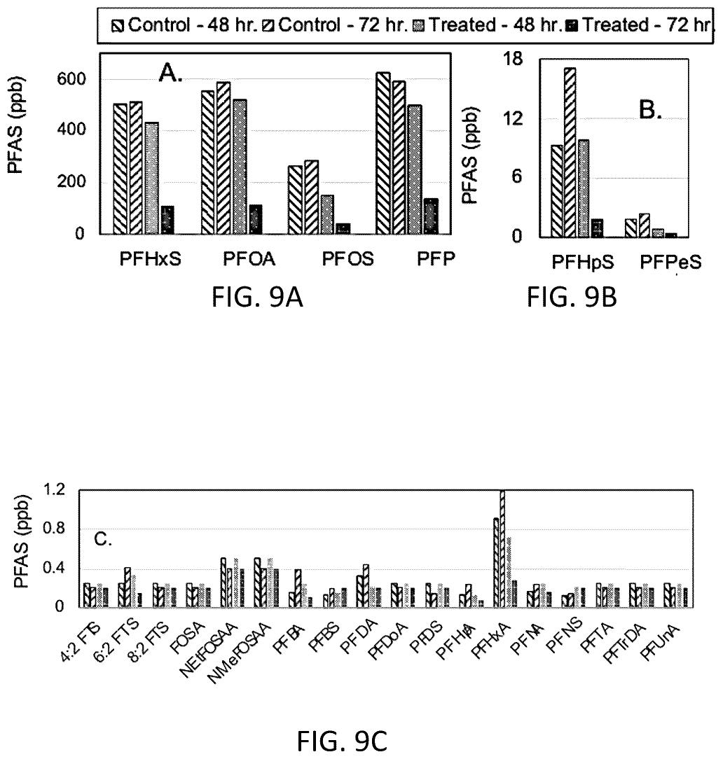

Four forms of PFAS compounds (PFHxS, PFOA, PFOS, and PFPeA) were added to water samples. Initial concentrations of the PFAS compounds ranged from 250 to 500 ppb in the water samples. The water samples were either left untreated or treated with Geobacillus miduosuji strain SH2B and maintained at 60° C. All water samples were otherwise exposed to the same conditions. Clay sorbent adsorbed PFAS from the contaminated water samples. The concentrations of the four added PFAS compounds and other PFAS compounds were analyzed by solid phase extraction and liquid chromatography/mass spectrometry with isotope dilution methods (EPA 537.1/533) after 48 hours and 72 hours of treatment. The total amount of the four added PFAS decreased by 18.4% and 79.6% after 48 and 72 hours relative to the controls respectively. The results are shown in FIG. 9A. Results using other PFAS are identified and shown in FIGS. 9B and 9C.

The identifications of the four columns for each PFAS are given in the figure legend. The full chemical name of the abbreviations used in the figure are given in Table 3 below.

TABLE 3

| Abbreviation | Analyte |
|---|---|
| 4:2FTS | 1H,1H,2H,2H-Perfluorohexane sulfonic acid |
| 6:2FTS | 1H,1H,2H,2H-Perfluorooctane sulfonic acid |
| 8:2FTS | 1H,1H,2H,2H-Perfluorodecane sulfonic acid |
| FOSA | perfluorooctane sulfonamide |
| NEtFOSAA | N-ethyl perfluorooctanesulfonamidoacetic acid |
| NMeFOSAA | N-methyl perfluorooctanesulfonamidoacetic acid |
| PFBA | Perfluorobutanoic acid |
| PFBS | Perfluorobutanesulfonic acid |
| PFDA | Perfluorodecanoic acid |
| PFDoA | Perfluorododecanoic acid |
| PFDS | perfluorodecane sulfonate, perfluorodecane sulfonic acid |
| PFHpA | Perfluoroheptanoic acid |
| PFHpS | Perfluoroheptanesulfonic acid |
| PFHxA | Perfluorohexanoic acid |
| PFHxS | Perfluorohexanesulfonic acid |
| PFNA | Perfluorononanoic acid |
| PFNS | perfluorononane sulfonate, perfluorononane sulfonic acid |
| PFOA | Perfluorooctanoic acid |
| PFOS | Perfluorooctanesulfonic acid |
| PFPeA | Perfluoropentanoic acid |
| PFPeS | Perfluoropentanesulfonic acid |
| PFTA | Perfluorotetradecanoic acid |
| PFTrDA | Perfluorotridecanoic acid |
| PFUnA | Perfluoroundecanoic acid |

### Example 2

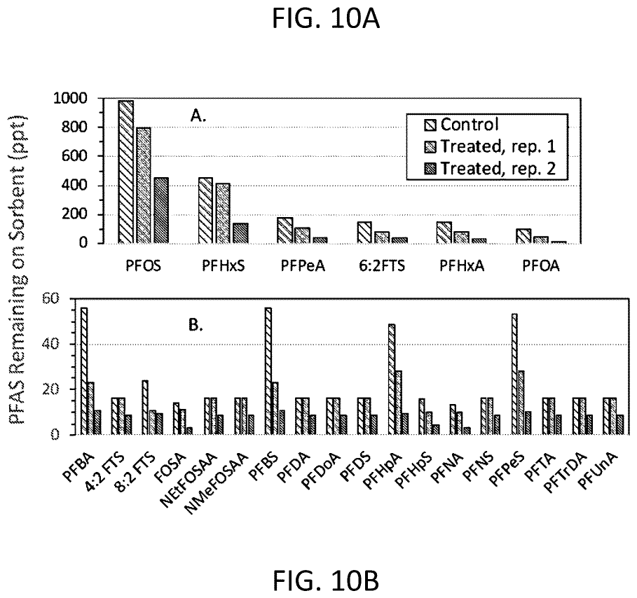

Additional tests were done with spent sorbent material containing PFAS indicating that when treated with Geobacillus miduosuji, the secondary waste streams represented by these sorbents also are treatable. Geobacillus midousuji ATCC Accession No. 55926 strain SH2B was added to test containers with spent sorbent material from an actual groundwater treatment site in Michigan. This spent sorbent material contained the adsorbed PFAS compounds derived from the contaminated groundwater. Additional containers were prepared as controls containing all of the same ingredients except the bacterium. The water was incubated at 60° C. for 72 hours. In this test with results show in in FIG. 10A, 6 of the PFAS compounds that represented over 80% of the total PFAS content (i.e., PFOS, PFHxS, PFPeA, 6:2 FTS, PFHxA, and PFOA) in the target analytical pool decreased by 24.1% and 64.5% in the 2 treated containers. Results using other PFAS are identified and shown in FIG. 10B.

### Example 3

PFOA was added to test containers containing water at a concentration of approximately 500 ppb. The water was treated with Geobacillus miduosuji SH2B and maintained at 60° C. A parallel control was done that was exposed to conditions similar in all respects to the treated samples except that the control did not contain Geobacillus miduosuji or any other bacteria. After 72 hours of treatment, the concentrations of PFOA were analyzed in 3 fractions of each container: the liquid supernatant, the pellet of Geobacillus miduosuji cells, and the volatiles generated in the head space of the sealed containers. Volatiles were collected on polyurethane foam (PUF) samples and Amberlite ion exchange resin. PFOA was extracted from the volatile collection material and pellet fractions and measured along with the supernatant fraction by solid phase extraction and liquid chromatography/mass spectrometry with isotope dilution methods (EPA 537.1/533). PFOA levels in the controls did not change. PFOA levels in the supernatant of the treated containers decreased and from 32% to 49% of the decrease in the supernatant was accounted for by PFOA in the cell pellet. The PFOA in the volatile fraction was insignificant in all containers. Overall, 36.1% and 21.9% of the original PFOA added to the treated containers was removed from the target analytical pool. The results are shown in Table 4.

TABLE 4

| | Replicate 1 | Replicate 2 |
|---|---|---|
| Control Fractions | | |
| Supernatant | 540 | 576 |
| Pellet | N.A. | N.A. |
| Volatiles | <5 | <5 |
| Treated Fractions | | |
| Supernatant | 267 | 311 |
| Cell Pellet | 93 | 120 |
| Volatiles | <5 | <5 |
| % Removal from target | 36.1% | 21.9% |
| pool by treatment | | |

### Example 4

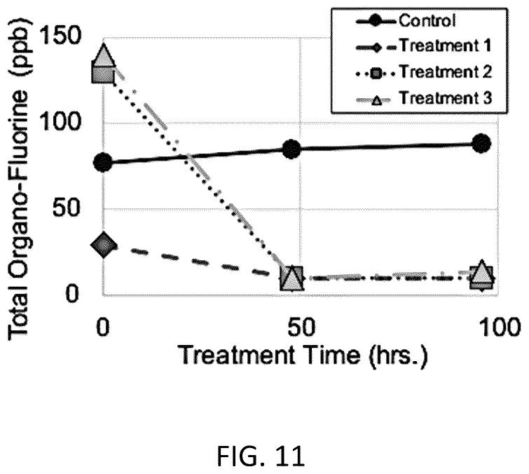

A solution of PFOs was prepared and was treated with Geobacillus miduosuji SH2B (T1, T2, and T3). Treatment with Geobacillus miduosuji was done for 96 hours in 3 containers in parallel with a fourth container similar all respects to the first except for the absence of G. miduosuji as a control. The total organo-fluorine pool (TOF), which estimates the total amount of carbon-fluorine compounds present was measured, with the results shown in FIG. 11.

### Example 5

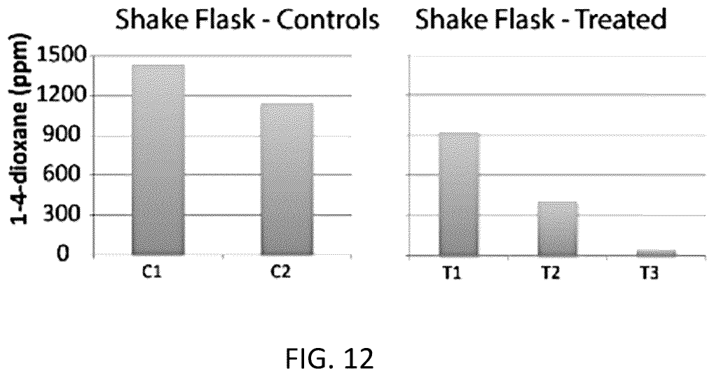

3 flasks (T1, T2, and T3) were loaded with an aqueous solution containing approximately 1000 ppm 1,4-dioxane (DX) and incubated for 8 hours with Geobacillus miduosuji SH2B. An additional 2 control flasks (C1 and C2) were prepared and tested under the same conditions but without bacteria. The 1,4-dioxane content in the resulting solution was analyzed by EPA method 8270D-SIM. At the end of the treatment, the concentration of 1,4-dioxane in the control flasks was 1230 ppm on average while that for the treated samples was 461 ppm. The results are shown in FIG. 12.

### Example 6

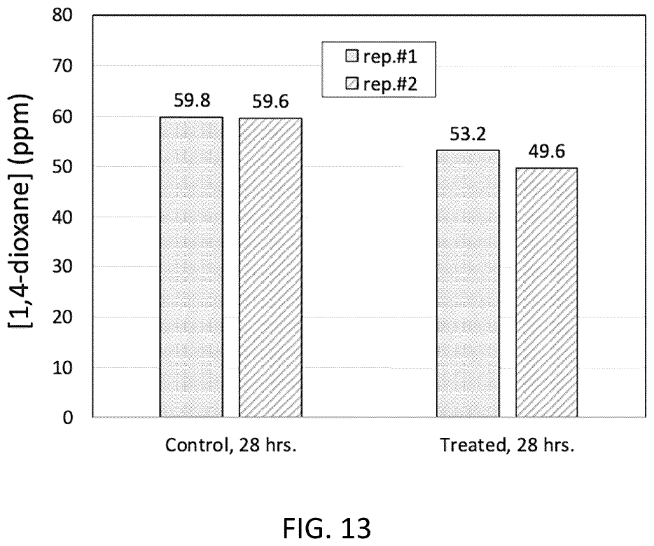

The experiment described in Example 5 was repeated but with (i) an aqueous solutions having a much lower initial 1,4-dioxane content (less than 10% of the initial 1,4-dioxane content in Example 5), (ii) only 2 flasks being treated with Geobacillus miduosuji SH2B, and (iii) the treatment time was 28 hours (instead of 8 hours). The results are shown in FIG. 13. After 28 hours, the treated samples contained on average 13.9% less 1,4-dioxane than the control samples.

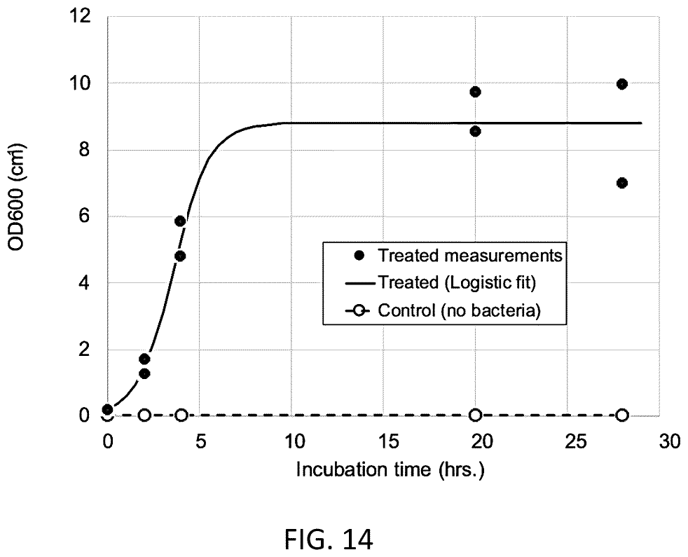

The growth of bacteria was monitored during this test by measuring the optical density of the solutions at 600 nm. FIG. 14 shows that the Geobacillus miduosuji grew as expected in the treated samples and there was no bacterial growth in the control samples, confirming that the difference in the results can be attributed to the growth of the bacteria.

### Example 7

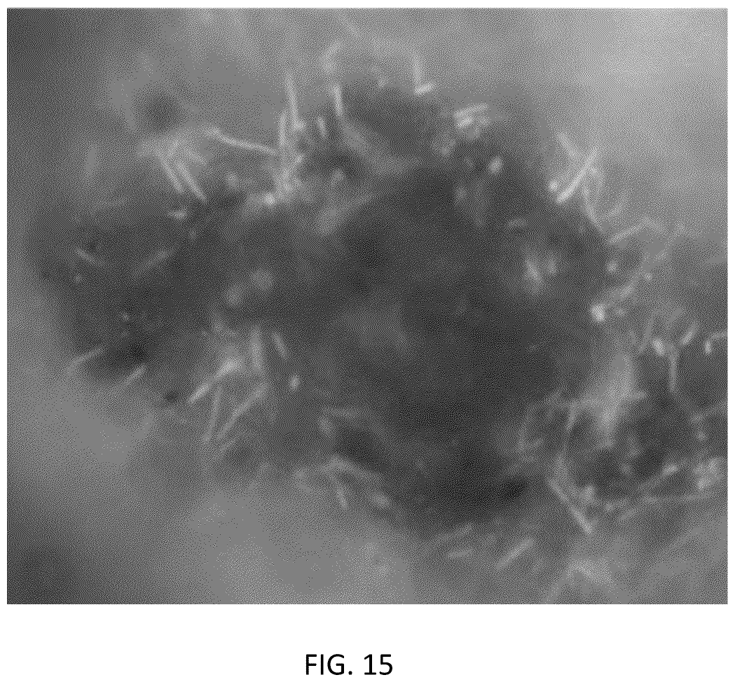

FIG. 15 shows biofilm formation by Geobacillus miduosujii. The microbial cells are visible as the green-fluorescent, rod-shaped structures in the photomicrograph. This mode of growth brings the strain in close physical contact with contaminants that are associated with particulate material. In addition, this biofilm facilitates the immobilization of the strain so that it can be used in a fixed bed bioreactor, entrapment in porous substances and encapsulation in microcapsules.

It should be understood that various combination, alternatives and modifications of the present invention could be devised by those skilled in the art. The present invention is intended to embrace all such alternatives, modifications and variances that fall within the scope of the appended claims.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

While the invention has been particularly shown and described with reference to a preferred embodiment thereof, it will be understood by those skilled in the art that various changes in form and details may be made therein without departing from the spirit and scope of the invention. Therefore, the scope of the invention is indicated by the appended claims, rather than by the foregoing description. All changes that come within the meaning and range of equivalency of the claims are to be embraced within their scope.

1. A method for bioremediation of soil, sediment, wastewater, or sorbent materials containing 1,4-dioxane and/or one or more PFAS compounds selected from perfluorohexane sulfonic acid (PFHxS), perfluorooctanoic acid (PFOA), perfluorooctane sulfonic acid (PFOS), perfluoropentanoic acid (PFPeA), perfluoroheptane sulfonic acid (PFHpS), perfluoropentane sulfonic acid (PFPeS), 4:2 fluorotelomer sulfonic acid (4:2 FTS), 6:2 fluorotelomer sulfonic acid (6:2 FTS), 8:2 fluorotelomer sulfonic acid (8:2 FTS), perfluorooctane sulfonamide (FOSA), N-ethyl perfluorooctane sulfonamidoacetic acid (NEtFOSAA), N-methyl perfluorooctane sulfonamidoacetic acid (NMeFOSAA), perfluorobutanoic acid (PFBA), perfluorobutane sulfonic acid (PFBS), perfluorodecanoic acid (PFDA), perfluorododecanoic acid (PFDoA), perfluorodecane sulfonic acid (PFDS), perfluoroheptanoic acid (PFHpA), perfluorohexanoic acid (PFHxA), perfluorononanoic acid (PFNA), perfluorononane sulfonic acid (PFNS), perfluorotetradecanoic acid (PFTA), perfluorotridecanoic acid (PFTrDA), and perfluoroundecanoic acid (PFUnA), the method comprising the steps of:
administering Geobacillus midousuji, or a mutant derived therefrom, to the soil, sediment, wastewater, or sorbent material to be bioremediated; and
incubating the Geobacillus midousuji in the soil, sediment, wastewater, or sorbent material at a temperature ranging from about 40 to about 70° C. and a humidity ranging from about 80% to about 100% for a period of time ranging from about 1 hour to about 20 days.

2. The method of claim 1, wherein the Geobacillus midousuji is strain SH2B (American Type Culture Collection (ATCC) No. 55926), strain SH2A (ATCC No. 202050), or mixtures thereof.

3. The method of claim 1, wherein the concentration of 1,4-dioxane or the PFAS compound in the soil, sediment or wastewater after incubation is less than about 100 parts per million (PPM).

4. The method of claim 3, wherein the concentration of 1,4-dioxane or the PFAS compound in the soil, sediment or wastewater after incubation is less than about 50 PPM.

5. The method of claim 4, wherein the concentration of 1,4-dioxane or the PFAS compound in the soil, sediment or wastewater after incubation is less than about 20 PPM.

6. The method of claim 1, wherein the Geobacillus midousuji to the soil, sediment, wastewater, or sorbent material ratio ranges from about 0.01% to about 1% w/w.

7. The method of claim 6, wherein the ratio ranges from about 0.01% to about 0.1% w/w.

8. The method of claim 6, wherein the ratio ranges from about 0.08% w/w to about 0.15% w/w.

9. The method of claim 8, wherein the ratio is about 0.1% w/w.

10. The method of claim 1, wherein the Geobacillus midousuji is activated into log phase growth before incubation with the soil, sediment, wastewater, or sorbent material by incubation of the Geobacillus midousuji under an aerobic condition at a temperature ranging from about 60° to about 65° C. in a medium comprising B-complex vitamin and amino-N with trace metals.

11. The method of claim 1, further comprising the step of rotating the soil, sediment, wastewater, or sorbent material at least about 1 to about 5 times per day during incubation with the Geobacillus midousuji.

12. The method of claim 1, wherein the Geobacillus midousujiis in the soil, sediment, wastewater, or sorbent material is inactivated.

13. The method of claim 1, wherein the incubation of the Geobacillus midousuji in the soil, sediment, wastewater, or sorbent material is done under aerobic conditions.

14. The method of claim 1, wherein the incubation of the Geobacillus midousuji in the soil, sediment, wastewater, or sorbent material is done under anaerobic conditions.

15. The method of claim 1, further comprising purifying or harvesting at least one protein product.

16. The method of claim 1, wherein the PFAS compound is selected from perfluorohexane sulfonic acid (PFHxS), perfluorooctanoic acid (PFOA), perfluorooctane sulfonic acid (PFOS), perfluoropentanoic acid (PFPeA), perfluoroheptane sulfonic acid (PFHpS), perfluoropentane sulfonic acid (PFPeS), 6:2 fluorotelomer sulfonic acid (6:2 FTS), perfluorobutanoic acid (PFBA), perfluorodecanoic acid (PFDA), perfluoroheptanoic acid (PFHpA), and perfluorohexanoic acid (PFHxA).

17. The method of claim 1, wherein the Geobacillus midousuji, or a mutant derived therefrom, is administered in the form of a biofilm.

18. The method of claim 1, wherein the Geobacillus midousuji, or a mutant derived therefrom, is administered in a stationary phase, in spore form, in planktonic form, in biofilm form, or a combination of planktonic and biofilm form.

19. The method of claim 1, wherein the administered Geobacillus midousuji, or a mutant derived therefrom, is in the form of a biofilm immobilized in a fixed film bioreactor.

20. A kit comprising:
Geobacillus midousuji or a mutant derived therefrom;
printed matter with instructions for activating the Geobacillus midousuji into log phase growth before incubation with soil, sediment, wastewater, or sorbent material by incubation of the Geobacillus midousuji under aerobic conditions at a temperature ranging from about 60° to about 65° C. in a medium comprising B-complex vitamin and amino-N with trace metals; and,
instructions for use of the Geobacillus midousuji in soil, sediment, wastewater, or sorbent material containing 1,4-dioxane and/or one or more PFAS compound selected from perfluorohexane sulfonic acid (PFHxS), perfluorooctanoic acid (PFOA), perfluorooctane sulfonic acid (PFOS), perfluoropentanoic acid (PFPeA), perfluoroheptane sulfonic acid (PFHpS), perfluoropentane sulfonic acid (PFPeS), 4:2 fluorotelomer sulfonic acid (4:2 FTS), 6:2 fluorotelomer sulfonic acid (6:2 FTS), 8:2 fluorotelomer sulfonic acid (8:2 FTS), perfluorooctane sulfonamide (FOSA), N-ethyl perfluorooctane sulfonamidoacetic acid (NEtFOSAA), N-methyl perfluorooctane sulfonamidoacetic acid (NMeFOSAA), perfluorobutanoic acid (PFBA), perfluorobutane sulfonic acid (PFBS), perfluorodecanoic acid (PFDA), perfluorododecanoic acid (PFDoA), perfluorodecane sulfonic acid (PFDS), perfluoroheptanoic acid (PFHpA), perfluorohexanoic acid (PFHxA), perfluorononanoic acid (PFNA), perfluorononane sulfonic acid (PFNS), perfluorotetradecanoic acid (PFTeDA), perfluorotridecanoic acid (PFTrDA), and perfluoroundecanoic acid (PFUnA), wherein the soil, sediment, wastewater, or sorbent material is maintained at a temperature ranging from about 40° C. to about 70° C. and a humidity ranging from about 90% to about 100% for a period of time ranging from about 1 hour to about 20 days.

21. (canceled)
